(19) United States
(12) Patent Application Publication
(10) Pub. No.: US 2007/0088433 A1
(43) Pub. Date: Apr. 19, 2007

(54) Accommodating intraocular lens system utilizing direct force transfer from zonules and method of use

(75) Inventors: Victor Esch, Albuquerque, NM (US); Terry Smiley, San Francisco, CA (US); Barry Cheskin, Mountain View, CA (US); Patrick Myall, San Francisco, CA (US); Bill Evans, San Francisco, CA (US); Henry Wu, Diamond Bar, CA (US); John Scholl, Danville, CA (US)

Correspondence Address: LUCE, FORWARD, HAMILTON & SCRIPPS LLP, 11988 EL CAMINO REAL, SUITE 200, SAN DIEGO, CA, 92130, US

(73) Assignee: PowerVision, Belmont, CA (US)

(21) Appl. No.: 11/253,031

(22) Filed: Oct. 17, 2005

(51) Int. Cl. A61F 2/16

(52) U.S. Cl. 623/6.13; 623/6.37; 623/6.51; 623/6.54

(57) ABSTRACT

An accommodating intraocular lens is provided having optical parameters that are altered in-situ, wherein an optic portion of the lens includes a lens piston that alters the shape of a lens element of the lens to alter the optical power of the lens, responsive to forces applied to a haptic portion to the lens by contraction of the ciliary muscles. Forces applied to the haptic portion are transferred hydraulically to cause the lens to become more or less accommodated. The haptic portion is retained in a fixed unaccommodated state during an initial healing period following implantation to facilitate affixation of the haptic portion to the capsule.

### FIELD OF THE INVENTION

The present invention relates to intraocular lenses (“IOLs”) having optical parameters that are changeable in-situ. More particularly, the invention has applications in IOLs for in-capsule implantation for cataract patients, wherein forces applied by the movement of the zonules induce movement of fluid media within the interior of the IOL, thereby altering an optical power of the lens to provide accommodation.

### BACKGROUND OF THE INVENTION

Cataracts are a major cause of blindness in the world and the most prevalent ocular disease. Visual disability from cataracts accounts for more than 8 million physician office visits per year. When the disability from cataracts affects or alters an individual's activities of daily living, surgical lens removal with intraocular lens (IOL) implantation is the preferred method of treating the functional limitations. In the United States, about 2.5 million cataract surgical procedures are performed annually, making it the most common surgery for Americans over the age of 65. About 97 percent of cataract surgery patients receive intraocular lens implants, with the annual costs for cataract surgery and associated care in the United States being upwards of $4 billion.

A cataract is any opacity of a patient's lens, whether it is a localized opacity or a diffuse general loss of transparency. To be clinically significant, however, the cataract must cause a significant reduction in visual acuity or a functional impairment. A cataract occurs as a result of aging or secondary to hereditary factors, trauma, inflammation, metabolic or nutritional disorders, or radiation. Age related cataract conditions are the most common.

In treating a cataract, the surgeon removes the crystalline lens matrix from the lens capsule and replaces it with an intraocular lens (“IOL”) implant. The typical IOL provides a selected focal length that allows the patient to have fairly good distance vision. Since the lens can no longer accommodate, however, the patient typically needs glasses for reading.

The imaging properties of the human eye are facilitated by several optical interfaces. A healthy youthful human eye has a total power of approximately 59 diopters, with the anterior surface of the cornea (e.g. the exterior surface, including the tear layer) providing about 48 diopters of power, while the posterior surface provides about −4 diopters. The crystalline lens, which is situated posterior of the pupil in a transparent elastic capsule supported by the ciliary muscles, provides about 15 diopters of power, and also performs the critical function of focusing images upon the retina. This focusing ability, referred to as “accommodation,” enables imaging of objects at various distances.

The power of the lens in a youthful eye can be adjusted from 15 diopters to about 29 diopters by adjusting the shape of the lens from a moderately convex shape to a highly convex shape. The mechanism generally accepted to cause this adjustment is that ciliary muscles supporting the capsule (and the lens contained therein), move between a relaxed state (corresponding to the moderately convex shape) to a contracted state (corresponding to the highly convex shape). Because the lens itself is composed of viscous, gelatinous transparent fibers, arranged in an “onion-like” layered structure, forces applied to the capsule by the ciliary muscles cause the lens to change shape.

Isolated from the eye, the relaxed capsule and lens take on a spherical shape. Within the eye, however, the capsule is connected around its circumference by approximately 70 tiny ligament fibers to the ciliary muscles, which in turn are attached to an inner surface of the eyeball. The ciliary muscles that support the lens and capsule therefore are believed to act in a sphincter-muscular mode. Accordingly, when the ciliary muscles are relaxed, the capsule and lens are pulled about the circumference to a larger diameter, thereby flattening the lens, whereas when the ciliary muscles are contracted, the lens and capsule relax somewhat and assume a smaller diameter that approaches a more spherical shape, thereby changing the diopter power of the lens.

As noted above, the youthful eye has approximately 14 diopters of accommodation. As a person ages, the lens hardens and becomes less elastic, so that by about age 45-50, accommodation is reduced to about 2 diopters. At a later age the lens may be considered to be non-accommodating, a condition known as “presbyopia”. Because the imaging distance is fixed, presbyopia typically entails the need for bi-focals to facilitate near and far vision.

Apart from age-related loss of accommodation ability, such loss is innate to the placement of IOLs for the treatment of cataracts. IOLs are generally single element lenses made from a suitable polymer material, such as acrylics or silicones. After placement, accommodation is no longer possible, although this ability is typically already lost for persons receiving an IOL. There is significant need to provide for accommodation in IOL products so that IOL recipients will have accommodating ability.

Although previously known workers in the field of accommodating IOLs have made some progress, the relative complexity of the methods and apparatus developed to date have prevented widespread commercialization of such devices. Previously known devices have proved too complex to be practical to construct or have achieved only limited success, due to the inability to provide accommodation of more than 1-2 diopters.

U.S. Pat. No. 5,443,506 to Garabet describes an accommodating fluid-filled lens wherein electrical potentials generated by contraction of the ciliary muscles cause changes in the index of refraction of fluid carried within a central optic portion. U.S. Pat. No. 4,816,031 to Pfoff discloses an IOL with a hard PMMA lens separated by a single chamber from a flexible thin lens layer that uses microfluid pumps to vary a volume of fluid between the PMMA lens portion and the thin layer portion and provide accommodation. U.S. Pat. No. 4,932,966 to Christie et al. discloses an intraocular lens comprising a thin flexible layer sealed along its periphery to a support layer, wherein forces applied to fluid reservoirs in the haptics vary a volume of fluid between the layers to provide accommodation.

Although fluid-actuated mechanisms such as described in the aforementioned patents have been investigated, commercially available accommodating lenses, such as developed by Eyeonics, Inc. of Aliso Viejo, Calif., rely on ciliary muscle contraction of the IOL haptics to vault the optic towards or away from the retina to adjust the focus of the device.

U.S. Patent Publication No. US2005/0119740, the application for which is co-pending and commonly assigned, describes an accommodating IOL in which shape changes of the capsular bag impose forces on a haptic portion of the IOL that in turn induce movement of fluid within an optic portion of the IOL. In the IOL described in that application, the lens assumes an accommodated state when unstressed, and moves to an unaccommodated state when subjected to laterally compressive forces by the capsule. While the IOLs described in that application include various mechanisms for retaining the capsule relatively taut throughout the range of accommodation, those IOLs do not provide a mechanism to ensure that the haptic portion does not migrate or become displaced when the ciliary muscles relax.

In view of the foregoing, it would be desirable to provide apparatus and methods that restore appropriate optical focusing power action to the human eye.

It further would be desirable to provide methods and apparatus wherein a dynamic lens surface may be effectively manipulated by the ciliary muscular mechanisms within the eye.

It still further would be desirable to provide methods and apparatus that utilize pressure applied by the accommodating muscular action to obtain a volumetric mechanical advantage in deflecting an optical surface of the IOL. In particular, it would be desirable to provide an IOL in which muscular pressure may be applied through one or more actuators to obtain such volumetric mechanical advantage.

It also would be desirable to provide an accommodating IOL having a feature that permits the haptic portion to be directly acted upon by movement of the zonules, so that radial movements of the zonules resulting from contraction or relaxation of the ciliary muscles are directly transferred to the haptic portion.

It further would be desirable to provide an accommodating IOL having a feature that permits the haptic portion to become affixed within the capsule, thereby enhancing resistance to migration or displacement of the lens during normal movements of the components of the eye associated with accommodation.

### SUMMARY OF THE INVENTION

In view of the foregoing, it is an object of the present invention to provide apparatus and methods that restore appropriate optical focusing power action to the human eye.

It is a further object of this invention to provide methods and apparatus wherein a dynamic lens surface may be effectively manipulated by the ciliary muscular mechanisms within the eye.

It is another object of the present invention to provide methods and apparatus that utilize pressure applied by the accommodating muscular action to obtain volumetric mechanical advantage in deflecting an optical surface of the IOL.

It is a further object of this invention to provide methods and apparatus for reversibly applying muscular pressure, through one or more actuators, to obtain a volumetric mechanical advantage in altering the optical parameters of one of more surfaces of the IOL.

It is also an object of the present invention to provide an accommodating IOL having a feature that permits the haptic portion to be directly acted upon by movement of the zonules, so that radial movements of the zonules resulting from contraction or relaxation of the ciliary muscles are directly transferred to the haptic portion.

It is a further object of the present invention to provide an accommodating IOL having a feature that permits the haptic portion to become affixed within the capsule, thereby enhancing resistance to migration or displacement of the lens during normal movements of the components of the eye associated with accommodation.

These and other objects of the present invention are accomplished by providing an intraocular lens responsive to variations in capsule shape and/or forces exerted by the ciliary muscle to actuate one or more haptic pistons. The haptic pistons are coupled to a lens piston that deflects a surface of the lens, e.g., from a moderately convex to a highly convex shape.

Further in accordance with the principles of the present invention, the lens is configured to promote affixation of a haptic portion of the lens to the capsular equator, thereby permitting risk force transfer between the zonules and haptic portion, and reducing the risk of migration or displacement of the IOL associated with operation of the accommodative mechanisms of the eye. Preferably, the lens is implanted into the eye restrained in a fixed unaccommodated state that urges the haptic portion into engagement with the capsular equator to promote the growth of fibrous tissue, thereby affixing the haptic portion to the capsule. Subsequently the restraint is removed to allow a complete range of accommodative motion of the lens.

In a preferred embodiment, the intraocular lens comprises an optic portion and a haptic (or non-optic) portion. The optic portion comprises a light transmissive substrate defining one or more fluid channels, one or more lens pistons coupled in fluid communication with the fluid channels, and anterior and posterior lens elements. One of the anterior and posterior lens elements includes a deflectable surface that is operatively coupled to the one or more lens pistons so that movement of the lens pistons causes the anterior or posterior lens to deflect. The other of the anterior or posterior lens elements may be coupled to the substrate or integrally formed therewith.

The haptic portion is disposed at the periphery of the optic portion and may comprise one or more arms that extend outward from the optic portion, each arm operatively coupled to a fluid channel in the optic portion. Each arm of the haptic portion includes a portion that engages the interior of the capsule and/or ciliary muscle, so that movement of the capsule and/or ciliary muscle is communicated via the fluid channels to the one or more lens pistons. In accordance with one aspect of the present invention, the haptic portion is biased to maintain the lens piston in an accommodated state. For such embodiments, relaxation of the ciliary muscle causes the zonules to transition the capsule to a less convex, unaccommodated shape. The capsule thereby applies tensile forces that deform the haptic portion and reduce fluid pressure in the lens piston, thereby causing the lens to transition to the unaccommodated state.

Alternatively, the lens piston may not be pressurized when the transducer is in the undeformed state. In this latter case, the lens may be configured so that contraction of the ciliary muscle induces thickening near the capsular equator, which in turn compresses the haptic portion to pressurize the lens piston and transition the lens to the accommodated state.

Methods of making and using the lens of the present invention also are provided.

### BRIEF DESCRIPTION OF THE DRAWINGS

Further features of the invention, its nature and various advantages will be more apparent from the accompanying drawings and the following detailed description of the preferred embodiments, in which:

FIG. 1 is a sectional side view of a human eye;

FIGS. 2A and 2B are, respectively, sectional side views of the lens and supporting structures of FIG. 1 illustrating relaxed and contracted states of the ciliary muscles;

FIGS. 3A and 3B are, respectively, an exploded perspective view and side sectional view, taken along line 3B-3B of FIG. 3A, of an exemplary accommodation mechanism suitable for use in the intraocular lens of the present invention;

FIG. 4 is a perspective view of an alternative embodiment of lens pistons suitable for use in the intraocular lens of FIG. 3;

FIGS. 5A and 5B are, respectively, side sectional views of the haptic portion of the lens of FIG. 3 in the accommodated and unaccommodated states;

FIGS. 6A-6C are, respectively, a perspective view of the lens of FIG. 3 disposed in a human eye and side sectional views of the lens in the accommodated and unaccommodated states;

FIGS. 7A-7C are, respectively, side sectional, exploded perspective, and plan views of an embodiment of the intraocular lens of the present invention;

FIGS. 8A-8C are respectively, side sectional, exploded perspective, and plan views of an alternative embodiment of the intraocular lens of the present invention; and

FIGS. 9A-9C are respectively, side sectional, exploded perspective, and plan views of an alternative embodiment of the intraocular lens of the present invention.

### DETAILED DESCRIPTION OF THE INVENTION

In accordance with the principles of the present invention, an intraocular lens is provided having a haptic portion and a light-transmissive optic portion. The optic portion contains one or more fluid-mediated pistons arranged to apply a deflecting force on an anterior or posterior element of the lens to provide accommodation of the lens. As used herein, the lens is fully “accommodated” when it assumes its most highly convex shape, and fully “unaccommodated” when it assumes its most flattened, least convex state. The lens of the present invention is capable of dynamically assuming any desired degree of accommodation between the fully accommodated state and fully unaccommodated state responsive to the movement of the ciliary muscles and deformation of the capsule.

Forces imposed on the haptic portion are applied to a transducer and communicated to one or more lens pistons that control deflection of an anterior or posterior element of the lens, resulting in a larger dynamic range of accommodation than heretofore is believed to have been available. The lens piston and surrounding fluids all are index-matched to prevent the occurrence of optical aberrations or reflections throughout the range of motion of the lens piston.

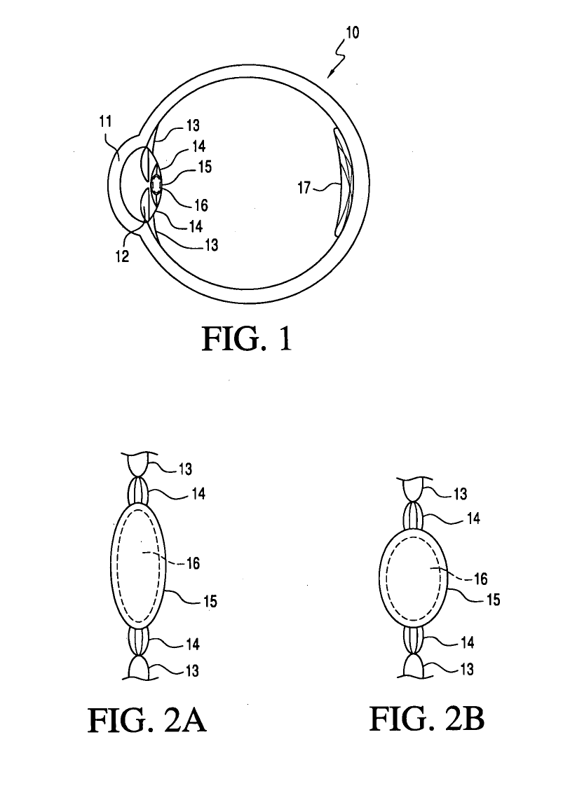

Referring to FIGS. 1 and 2, the structure and operation of a human eye are first described as context for the present invention. Eye 10 includes cornea 11, iris 12, ciliary muscles 13, ligament fibers or zonules 14, capsule 15, lens 16 and retina 17. Natural lens 16 is composed of viscous, gelatinous transparent fibers, arranged in an “onion-like” layered structure, and is disposed in transparent elastic capsule 15. Capsule 15 is joined by zonules 14 around its circumference to ciliary muscles 13, which are in turn attached to the inner surface of eye 10. Vitreous 18 is a thick, transparent substance that fills the center of eye 10.

Isolated from the eye, the relaxed capsule and lens takes on a spherical shape. However, when suspended within the eye by zonules 14, capsule 15 moves between a moderately convex shape (when the ciliary muscles are relaxed) to a highly convex shape (when the ciliary muscles are contracted). As depicted in FIG. 2A, when ciliary muscles 13 relax, capsule 15 and lens 16 are pulled about the circumference, thereby flattening the lens. As depicted in FIG. 2B, when ciliary muscles 13 contract, capsule 15 and lens 16 relax and become thicker. This allows the lens and capsule to assume a more spherical shape, thus increasing the diopter power of the lens.

Currently available accommodating lenses, such as the Crystalens device developed by Eyeonics, Inc., Aliso Viejo, Calif., converts ciliary muscle movements into vaulting movements of an optic portion of the IOL. Devices such as the Crystalens thus do not employ the natural accommodation mechanisms described above.

By contrast, according to one aspect of the present invention, an intraocular lens is designed to engage capsule 15 and to transition between the accommodated and unaccommodated states responsive to forces applied to capsule 15 by ciliary muscle 13 and zonules 14, thereby more closely mimicking operation of the natural eye. More preferably, the haptic portion is configured to be affixed to the capsular equator, as described herein below.

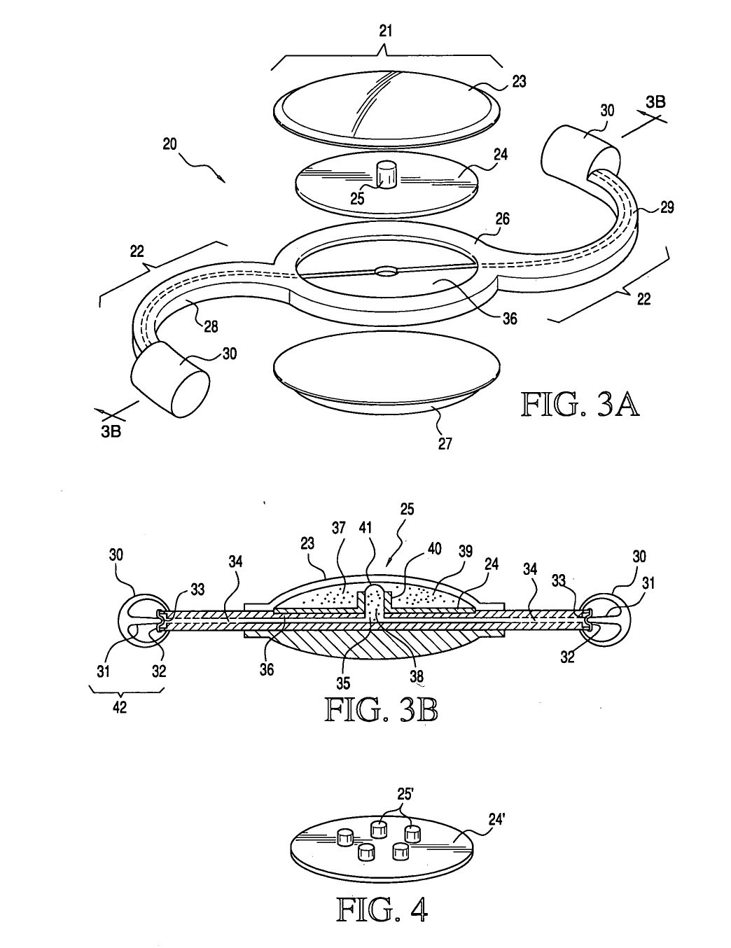

Referring to FIGS. 3 to 6, the intraocular lens of U.S. Patent Publication No. US2005/0119740 is described as providing a suitable accommodation mechanism for use with the intraocular lens of the present invention. That publication describes an accommodating IOL in which shape changes of the capsular bag impose forces on a haptic portion of the IOL that in turn induce movement of fluid within an optic portion of the IOL. The lens assumes an accommodated state when unstressed, and transitions to an unaccommodated state when subjected to laterally compressive forces by the capsule.

More specifically, referring to FIGS. 3A and 3B, an exemplary embodiment of an intraocular lens suitable for implementing the present invention is described. IOL 20 comprises optic portion 21 and haptic portion 22. Optic portion 21 is constructed of light transmissive materials, while haptic portion 22 is disposed at the periphery of the optic portion and does not participate in focusing light on the retina of the eye.

Optic portion 21 comprises anterior lens element 23, actuator layer 24 including lens piston 25, substrate 26 and posterior lens element 27, all made of light-transmissive materials, such as silicone or acrylic polymers or other biocompatible materials as are known in the art of intraocular lenses. Haptic portion 22 illustratively comprises arms 28 and 29 extending from substrate 26, although other haptic configurations may be employed. Each of arms 28 and 29 includes a transducer 30 and a haptic piston including force-concentrating fin 31, diaphragm 32 and reservoir 33. Reservoirs 33 are coupled in fluid communication with the interior of lens piston 25 via channels 34 that extend from the reservoirs 33 to well 35 disposed beneath lens piston 25.

In FIG. 3B, transducers 30 are shown in an undeformed state in which force-concentrating fins 31 apply a maximum deflection to diaphragms 32, thereby fully deflecting end wall 41 and driving anterior element 23 to the fully accommodated position. This corresponds to a fully-contracted state of the ciliary muscles, as described herein below.

Actuator layer 24 is disposed in recess 36 of substrate 26, and preferably comprises a sturdy elastomeric material. Actuator layer 24 isolates the fluid in channels 34, well 35 and the interior of lens piston 25 from the fluid disposed in the space 37 between anterior lens element 23 and actuator layer 24. Fluids 38 and 39 disposed, respectively, within channels 34 and space 37, preferably comprise silicone or acrylic oils and are selected to have refractive indices that match the materials of anterior lens element 23, actuator layer 24 and substrate 26.

In a preferred embodiment, lens piston 25 includes substantially nondeformable cylindrical side wall 40 coupled to expandable end wall 41. End wall 41 is configured to deflect outward responsive to pressure applied within sidewall 40 by fluid movement from the haptic portion. End wall 41 contacts the interior surface of anterior lens element 23, so that deflection of end wall 41 of the lens piston causes a corresponding deflection of anterior lens surface 23. Such deflections cause the anterior lens element to assume a spherical shape with a shorter radius of curvature, thereby changing the diopter power of the lens. As will of course be understood, optic portion could instead be arranged so that the lens piston deflects posterior lens element 27; the arrangement depicted in FIG. 3 is illustrative only.

The inner surface and thickness of anterior element 23 (relative to the optical axis of the lens) are selected so that the outer surface of anterior element 23 retains an optically corrective shape, e.g., spherical, throughout the entire range of motion of lens piston 25, e.g., for accommodations 0-10 diopters. It should of course be understood that the inner surface and thickness of anterior element 23 may be selected to provide an aspherical outer surface, as required for a desired degree of optical correction.

As shown in FIG. 3, one preferred embodiment of actuator layer 24 includes a single lens piston 25 located at the center of optic portion 21. Alternative embodiments of actuator layer 24′ may include an array of lens pistons 25′ spaced apart in a predetermined configuration on the anterior surface of the actuator layer, as depicted in FIG. 4, as may be required to impose a desired pattern of localized deflection on the anterior lens element. As will be apparent to one of skill in the art, an annular structure may be substituted for the individual lens pistons depicted in FIG. 4, and side walls 40 may be of any desired shape other than cylindrical.

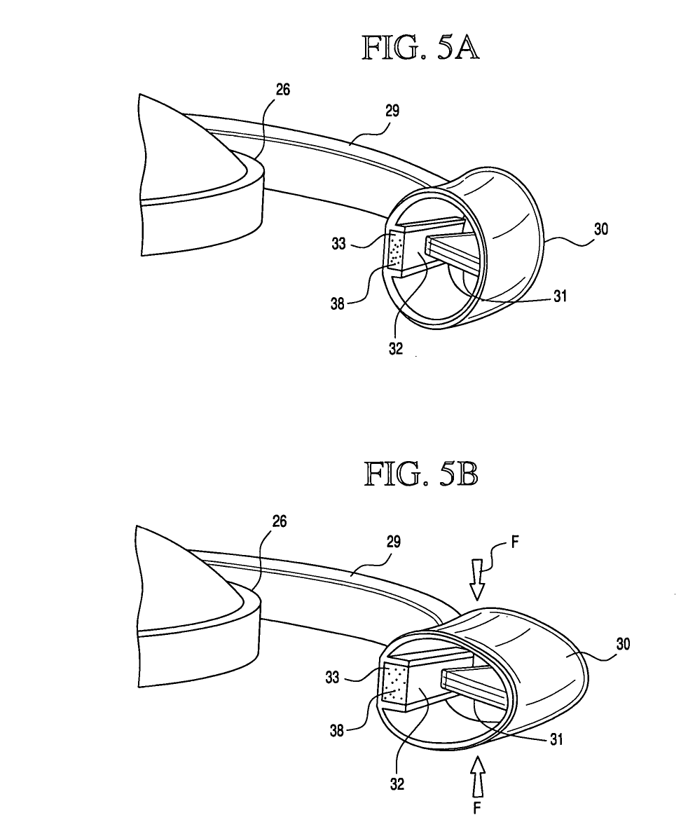

Referring now to FIGS. 5A and 5B, flexible and resilient transducers 30 support force-concentrating fins 31 biased against diaphragms 32. Each diaphragm 32 comprises an elastomeric cover for a corresponding reservoir 33 filled with fluid 38. As described herein above, fluid 38 communicates through channels 34 into well 35 and the interior of lens piston 25. Transducers 30 are constructed from a resilient, elastomeric material that changes shape responsive to forces applied by capsule 15 from the ciliary muscles 13 and zonules 14.

In FIG. 5A, the haptic piston is shown in an undeformed state (as in FIG. 3B), corresponding to the ciliary muscles being fully contracted. In this state, the apex of fin 31 bears against diaphragm 32 to develop the maximum force resulting from the bias of transducer 30. Inward displacement of diaphragm 32 in turn displaces fluid through channels 34 (see FIG. 3) to well 35, resulting in expansion of end wall 41 of lens piston 25. When transducer 30 is in the undeformed state, fin 31 displaces the maximum volume of fluid from the haptic portion to lens piston 25, resulting in the maximum deflection of anterior element 23, and thus the maximum degree of accommodation of the lens. This corresponds to the state in which the ciliary muscles are fully contracted, and zonules 14 and capsule 15 apply the least amount of compressive force to the anterior and posterior surfaces of transducer 30.

When the ciliary muscles relax, however, the tension in the zonules increases, causing capsule 15 to assume a less convex shape (see FIG. 2A) and the lens to transition to its unaccommodated state. When the capsule becomes taut, it applies compressive forces F to the anterior and posterior surfaces of transducer 30, causing the transducer to deform to the elliptical shape depicted in FIG. 5B. Deformation of transducers 30 moves fins 31 away from diaphragms 32, thereby unloading the diaphragms and reducing the fluid pressure applied to lens piston 25. This in turn permits lens piston 25 to move to an undeflected state, reducing deflection of anterior lens element 23 and returning the lens to an unaccommodated state.

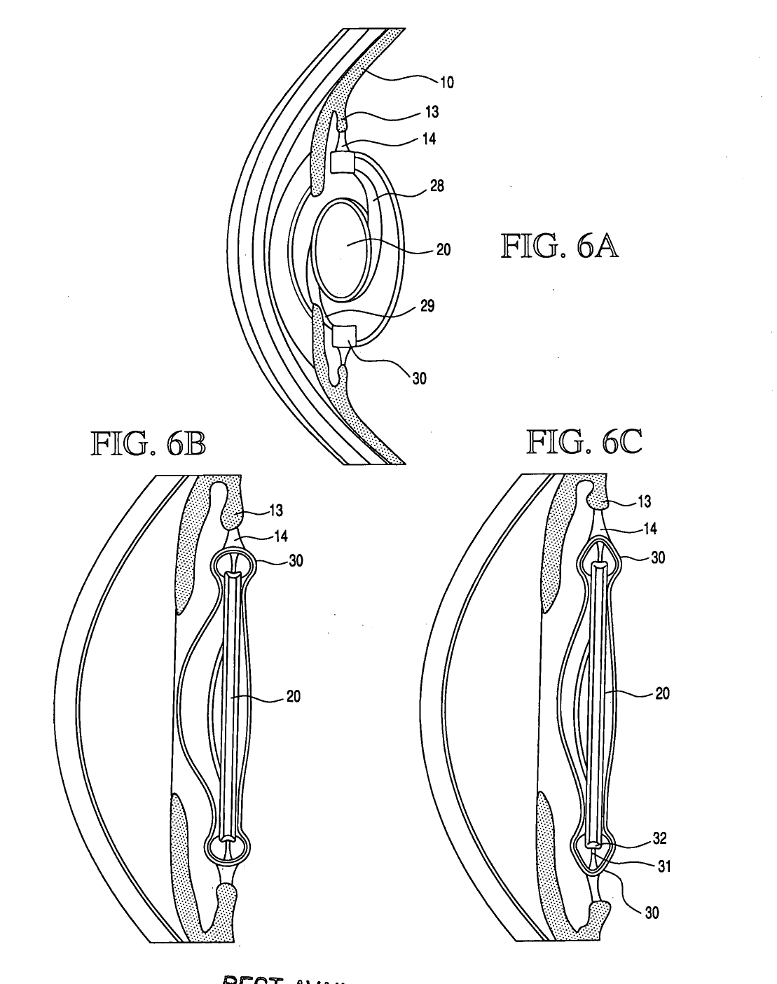

Referring now to FIGS. 6A to 6C, IOL 20 is shown implanted into capsule 15 of human eye 10. When so implanted, haptic arms 28 and 29 support the IOL within the capsule, while transducers 30 engage the interior of the capsule at locations adjacent to ciliary muscles 13. In FIG. 6B the ciliary muscles are shown in a contracted state, in which the compressive forces applied by zonules 14 and capsule 15 to transducers 30 is lowest and transducers 30 assume the undeformed position. This also corresponds to transducers 30 applying the least tension to capsule 15 and zonules 14. As discussed above, in the undeformed position, fins 30 are biased against diaphragms 32, displacing fluid 38 from reservoirs 33 to the lens piston. In FIG. 6C, the ciliary muscles are relaxed, and zonules 14 pull capsule 15 taut into a somewhat ellipsoidal shape. As noted above, in this state the capsule applies compressive forces to the lateral surfaces of transducers 30 that ensure that lens piston 25 is drawn to its fully retracted position.

The volume of fluid in the accommodating lens may be selected so that the forces required to provide a useable range of accommodation are satisfactory for a preselected population of patients. Alternatively, the volume of fluid used in IOL 20 may be specified during manufacture for a given patient, or may be adjusted prior to implantation of the IOL on a patient-by-patient basis. In this manner, the forces developed by lens piston 25 and the haptic pistons may be tailored for a specific patient. In addition, the number, shape and placement of lens pistons 25′ on actuator layer 24′ may be selected, e.g., prescribed during manufacture, to optimize accommodation of the lens for a specific patient.

The IOL described in FIGS. 3-6 above contemplates that the capsule will remain relatively taut throughout the range of accommodation. That IOL does not, however, provide direct transfer of forces from the zonules to the IOL, nor a mechanism to ensure that the haptic portion does not migrate or become displaced when the ciliary muscles relax. The improvements described hereinafter are intended to permit the haptic portion to be directly acted upon by movement of the zonules. In this manner, radial movements of the zonules resulting from contraction or relaxation of the ciliary muscles are directly transferred to the haptic portion. In addition, the improvements provided herein address the potential issue of migration of the intraocular lens within the capsular bag by affixing the arms of the haptic portion to the capsular equator.

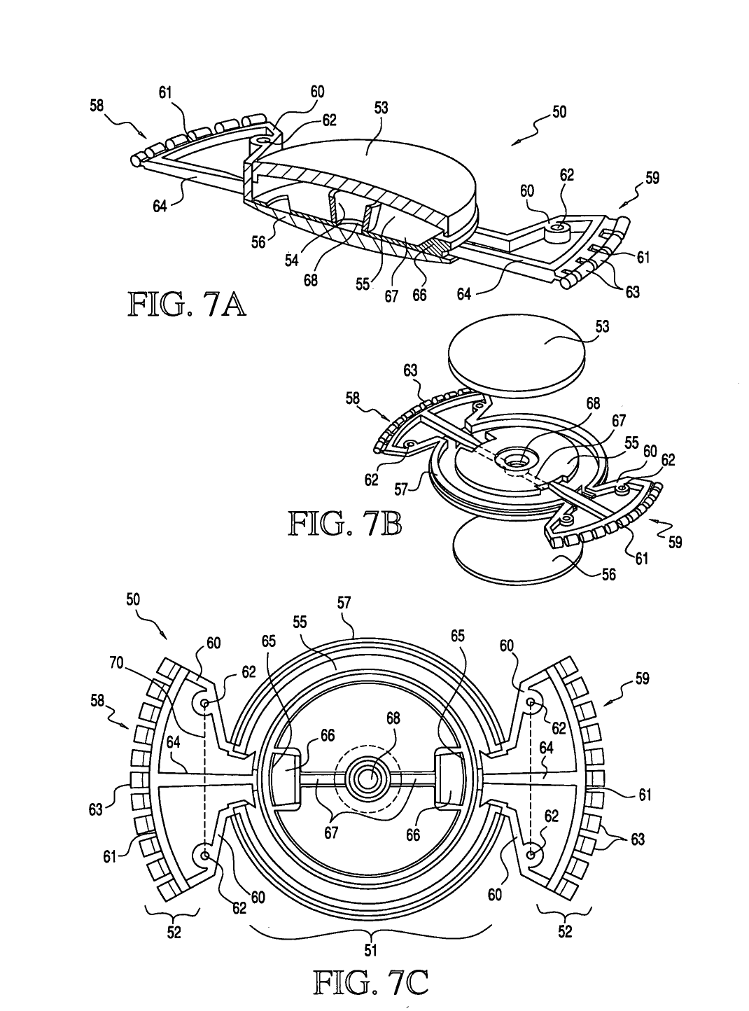

Referring now to FIGS. 7A-7C, an intraocular lens constructed in accordance with the principles of the present invention is described. IOL 50 comprises optic portion 51 and haptic portion 52. Optic portion 51 is constructed of light transmissive materials, while haptic portion 52 is disposed at the periphery of the optic portion and does not participate in focusing light on the retina of the eye.

Optic portion 51 comprises anterior lens element 53 with bellows-shaped lens piston 54, substrate 55 and posterior lens element 56, all made of light-transmissive materials, such as silicone or acrylic polymers or other biocompatible materials as are known in the art of intraocular lenses. As in the preceding embodiment, fluids having indices of refraction matched to the solid components of optic portion 51, such as silicone or acrylic oils, are disposed within the lens piston, channels and other spaces of the lens. The side wall of lens piston 54 is configured to deflect inward responsive to pressure increases within the lens piston, which in turn deflect anterior lens element 53 to assume more convex shape, thereby changing the diopter power of the lens.

The inner surface and thickness of anterior lens element 53 (relative to the optical axis of the lens) are selected so that the outer surface of anterior element 53 retains an optically corrective shape, e.g., spherical, throughout the entire range of motion of lens piston 54, e.g., for accommodations 0-10 diopters. It should of course be understood that the inner surface and thickness of anterior lens element 53 also may be selected to provide an aspherical outer surface, as required for a desired degree of optical correction.

Haptic portion 52 comprises support ring 57 carrying arms 58 and 59. Preferably, haptic portion 52 is molded of an elastic polymeric material, such as silicone or polyurethane, that tends to return to its undeformed shape after a deformation occurs. Other embodiments of haptic portion 52 may be machined from a single piece of shape memory material, such as nickel-titanium, or may be formed from other material having elastic properties. Support ring 57 surrounds and is affixed to substrate 55. Each of arms 58 and 59 comprises a pair of support elements 60 coupled to arched portion 61. Suture anchors 62 are disposed on support elements 60, while arched portion 61 carries outwardly-directed barbs 63 and inwardly-directed piston element 64.

As used herein, barbs may comprise one or more parallel ridges, hooks, or other attachment devices. These devices may be simple, such as a roughened surface of the haptic or use of a dimpled texture. Likewise, these devices may be more advanced, such as an arrangement of attachment devices in an offset fashion, resembling fish scales, to further enhance attachability and reduce the tendency for the haptic portion to dislodge from the capsular bag.

The radially inward-most ends of each of piston elements 64 is disposed in contact with diaphragm 65 and reservoir 66. Each of reservoirs 66 is coupled in fluid communication with the interior of lens piston 54 via channels 67 that extend from reservoirs 66 to well 68 disposed beneath lens piston 54. Together, piston elements 64, diaphragm 65 and reservoir 66 form a haptic piston that supplies fluid to lens piston 54 responsive to movements of the ciliary muscle and the capsular bag; these fluid movements in turn transition lens 50 between the accommodated and unaccommodated states.

Haptic portion 52 is configured so that in its unstressed state piston elements 64 extend to radially inward to the maximum extent. This state corresponds to maximum displacement of fluid from the haptic piston to lens piston 54, maximum displacement of lens piston 54, and the fully accommodated state of lens 50. Barbs 63 are configured to engage the capsular equator so that when the ciliary muscles relax, the capsule pulls arched portions 61 and piston elements 64 radially outward, thereby transitioning the lens to the unaccommodated state.

In accordance with one aspect of the present invention, IOL 50 is configured to be placed in the capsule of a patient's eye in the unaccommodated state, with the arched portions of arms 58 and 59 extending radially outward to the maximum extent. This may be achieved by placing suture 70 (one shown in dotted line in FIG. 7C) across suture anchors 62 to compress the support elements 60 of each arm 58 and 59 towards one another. This in turn causes arched portions 61 to be deflected radially outward and holds piston elements 64 out of contact with diaphragms 65 of the haptic pistons.

When IOL 50 is implanted into the patient's native capsule in the unaccommodated state, barbs 63 engage the capsular equator. Sutures 70 remain in place during an initial period of several days to weeks, during which time fibrous tissue adheres to arched portions 61 and barbs 63, causing arms 58 and 59 to become affixed to the capsular equator. Preferably, sutures 70 comprise a resorbable material that disintegrates after several days to weeks, thereafter releasing arms 58 and 59 to allow IOL 50 to accommodate responsive to movements of the ciliary muscles and capsule.

More specifically, once sutures 70 are resorbed, support elements 61 no longer retain with arched portions 61 at the maximum radially outward deflection. Accordingly, arms 58 and 59 are free to move inward under the bias of the predetermined shape stored in the shape memory material until constrained by the opposing forces applied by the ciliary muscles, capsule and zonules. For example, if the ciliary muscles contract, the capsular equator moves radially inward and piston elements 64 bear fully against diaphragms 65. This action displaces fluid from reservoirs 66 to lens piston 54, causing the anterior lens element to deflect to the accommodated state. When the ciliary muscles contract, the zonules pull the capsular equator to a larger diameter, thereby applying tensile forces to arms 58 and 59 that reduce the inward deflection of piston elements 64. This action causes fluid in lens piston 54 to move to reservoirs 66, and reduces the degree of accommodation provided by IOL 50.

In a preferred embodiment, the resorbable characteristics of the suture may be selected to correspond to the expected “heal-in” period, that is, the period during which fibrous growth cause barbs 63 to become embedded in the capsular equator. Thus, the sutures will naturally resorb and release arms 58 and 59, thereby allowing accommodating movement of lens piston 54 and anterior element 53. In an alternative embodiment the sutures may be released by severing the sutures using a Nd:YAG laser or similar device.

As for the preceding embodiment, the volume of fluid in IOL 50 may be selected so that the forces required to provide a useable range of accommodation are satisfactory for a preselected population of patients. Alternatively, the volume of fluid used in the IOL may be specified during manufacture for a given patient, or may be adjusted prior to implantation of the IOL on a patient-by-patient basis. In this manner, the forces developed by lens piston 54 and the haptic pistons may be tailored for a specific patient.

As will of course be understood, optic portion 51 could instead be arranged so that the lens piston is integrated with posterior lens element 56 instead of anterior lens element 53, and causes deflection the posterior lens element 56; the arrangement depicted in FIG. 7 is illustrative only.

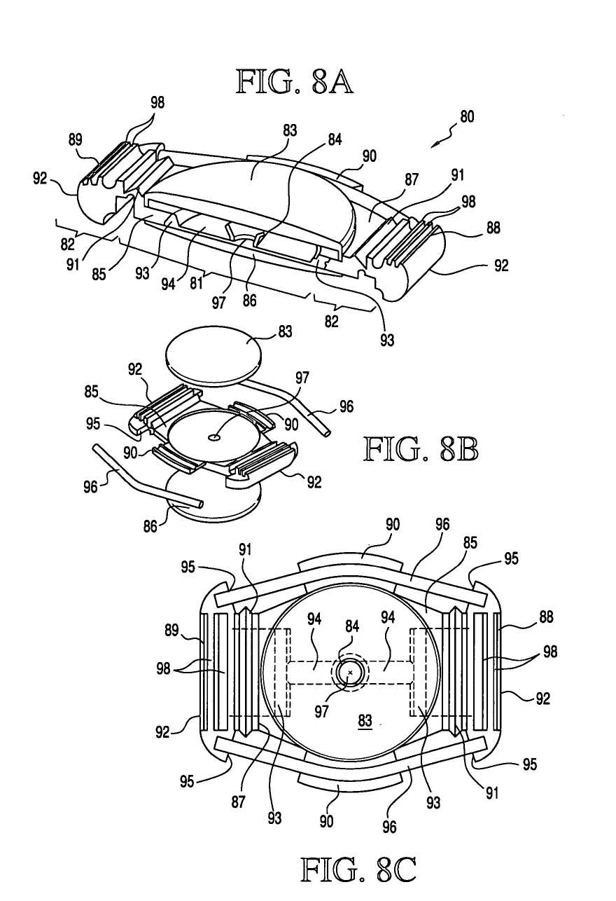

Referring now to FIGS. 8A-8C, an alternative embodiment of an intraocular lens of the present invention is described. IOL 80 comprises optic portion 81 and haptic portion 82. Optic portion 81 is constructed of light transmissive materials, while haptic portion 82 is disposed at the periphery of the optic portion and does not participate in focusing light on the retina of the eye.

Optic portion 81 comprises anterior lens element 83 with bellows-shaped lens piston 84, a portion of substrate 85 and posterior lens element 86, all made of light-transmissive materials, such as silicone or acrylic polymers or other biocompatible materials as are known in the art of intraocular lenses. As in the preceding embodiment, fluids having indices of refraction matched to the solid components of optic portion 81, such as silicone or acrylic oils, are disposed within the lens piston, channels and other spaces of the lens. The side wall of lens piston 84 is configured to deflect inward responsive to pressure increases within the lens piston, which in turn deflect anterior lens element 83 to assume more convex shape, thereby changing the diopter power of the lens.

The inner surface and thickness of anterior lens element 83 (relative to the optical axis of the lens) are selected so that the outer surface of anterior element 83 retains an optically corrective shape, e.g., spherical, throughout the entire range of motion of lens piston 84, e.g., for accommodations 0-10 diopters. It should of course be understood that the inner surface and thickness of anterior lens element 83 also may be selected to provide an aspherical outer surface, as required for a desired degree of optical correction.

Haptic portion 82 comprises peripheral portion 87 of substrate 85 including arms 88 and 89 and retainer supports 90. Preferably, haptic portion 82 is molded or machined from a single piece of a resilient elastomeric material. Each of arms 88 and 89 comprises flexible bellows 91 having ribbed distal portion 92 configured to engage the capsular equator. Bellows 91 are coupled to reservoirs 93 in substrate 85 so that fluid disposed in bellows 91 may be displaced through channels 94 to and from lens piston 84 responsive to movement of distal portions 92. Distal portions 92 in addition include recesses 95 that accept the ends of retainer rods 96 disposed in retainer supports 90, for the purposes described below.

More particularly, each of reservoirs 93 is coupled in fluid communication with the interior of lens piston 84 via channels 94 that extend from reservoirs 93 to well 97 disposed beneath lens piston 84. Together, bellows 91 and reservoirs 93 form haptic pistons that supply fluid to lens piston 84 responsive to movements of the ciliary muscle and the capsular bag; these fluid movements in turn transition lens 80 between the accommodated and unaccommodated states.

Haptic portion 82 is configured so that in its unstressed state bellows 91 extend to radially inward to the maximum extent. This state corresponds to maximum displacement of fluid from the haptic pistons to lens piston 84, maximum displacement of lens piston 84, and the fully accommodated state of lens 80. Ribs 98 on distal portions 91 are configured to engage the capsular equator so that when the ciliary muscles relax, the capsule pulls distal portions 92 and bellows 91 radially outward, thereby transitioning the lens to the unaccommodated state.

In accordance with one aspect of the present invention, IOL 80 is configured to be placed in the capsule of a patient's eye in the unaccommodated state, with arms 88 and 89 extending radially outward to the maximum extent. This is achieved by extending retainer rods 96 from retainer supports 90 so that the distal ends of the retainer rods 96 are captured in recesses 95 in distal portions 92 (as depicted in FIG. 8C). Retainer rods 96 preferably comprise a shape memory alloy having a low temperature elongated martensitic state and transform to a reduced length when heated into the austenitic phase. Retainer rods 96 therefore are installed in retainer supports 90 in the elongated state so that they are slidingly captured in recesses 95 to retain distal portions 92 at the maximum extension. When heated into the austenitic phase, retainer rods 96 undergo a phase transformation that results in retainer rods shortening and disengaging from recesses 95. In some preferred embodiments, retainer rods 96 shorten to the extent that the rods become fully retracted within the retainer supports 90.

When IOL 80 is implanted into the patient's native capsule in the unaccommodated state, ribs 98 engage the capsular equator. Retainer rods 96 are allowed to remain in the elongated state during an initial period of several days to weeks, during which time fibrous tissue adheres to distal portions 92 and ribs 98, causing arms 88 and 89 to become affixed to the capsular equator. Once distal portions 92 become adhered to the capsular equator, retainer rods 96 may be heated into the austenitic phase, for example, using a laser, RF or ultrasound energy, and shorten to release arms 88 and 89, thereby allowing IOL 80 to accommodate responsive to movements of the ciliary muscles and capsule.

Once retainer rods 96 are transformed to the reduced length state, bellows 91 are no longer held at the maximum outward extension. Accordingly, arms 88 and 89 are free to move inward under the bias of bellows 91 until constrained by the opposing forces applied by the ciliary muscles, capsule and zonules. For example, if the ciliary muscles contract, the capsular equator moves radially inward and distal portions 92 bear fully against bellows 91. This action displaces fluid from reservoirs 93 to lens piston 84, causing the anterior lens element to deflect to the accommodated state. When the ciliary muscles contract, the zonules pull the capsular equator to a larger diameter, thereby applying tensile forces to arms 88 and 89 that reduce the inward deflection of distal portions 92 and bellows 91. This action causes fluid in lens piston 84 to move to reservoirs 93, and reduces the degree of accommodation provided by IOL 80.

In a preferred embodiment, retainer rods 96 are left in the elongated state for a period selected to correspond to the expected “heal-in” period, that is, the period during which fibrous growth cause ribs 98 to become embedded in the capsular equator. Thereafter, the retainer rods are heated to release arms 88 and 89, thereby allowing accommodating movement of lens piston 84 and anterior element 83.

As for the preceding embodiments, the volume of fluid in IOL 80 may be selected so that the forces required to provide a useable range of accommodation are satisfactory for a preselected population of patients. Alternatively, the volume of fluid used in the IOL may be specified during manufacture for a given patient, or may be adjusted prior to implantation of the IOL on a patient-by-patient basis. In this manner, the forces developed by lens piston 84 and the haptic pistons may be tailored for a specific patient.

As will of course be understood, optic portion 81 could instead be arranged so that the lens piston is integrated with posterior lens element 86 instead of anterior lens element 83, and causes deflection the posterior lens element 86; the arrangement depicted in FIG. 8 is illustrative only.

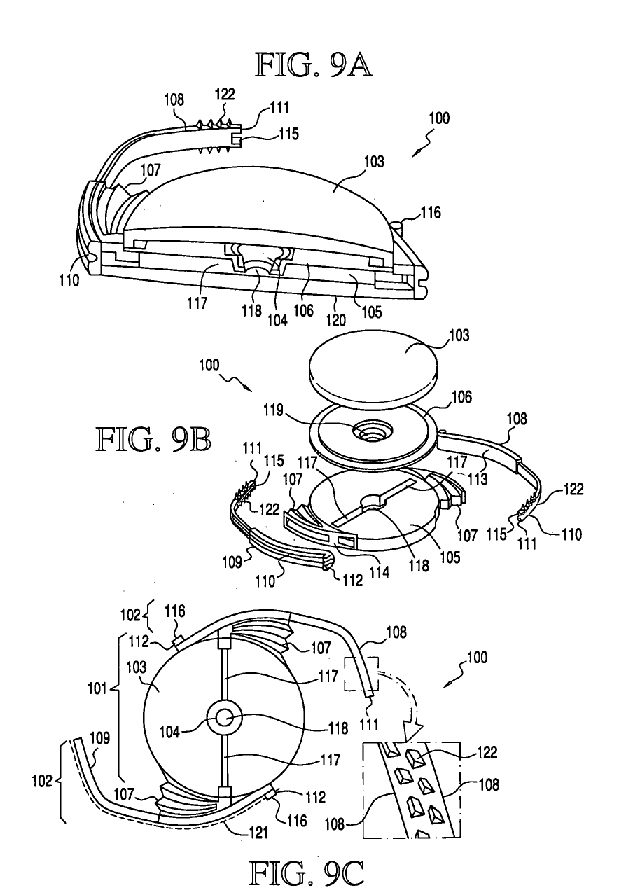

Referring now to FIGS. 9A-9C, a further alternative embodiment of an intraocular lens constructed in accordance with the principles of the present invention is described. IOL 100 comprises optic portion 101 and haptic portion 102. Optic portion 101 is constructed of light transmissive materials, while haptic portion 102 is disposed at the periphery of the optic portion and does not participate in focusing light on the retina of the eye.

Optic portion 101 comprises anterior lens element 103 with bellows-shaped lens piston 104, substrate 105 and intermediate layer 106, all made of light-transmissive materials, such as silicone or acrylic polymers or other biocompatible materials as are known in the art of intraocular lenses. As in the preceding embodiments, fluids having indices of refraction matched to the solid components of optic portion 101, such as silicone or acrylic oils, are disposed within the lens piston, channels and other spaces of the lens. The side wall of lens piston 104 is configured to deflect inward responsive to pressure increases within the lens piston, which in turn deflect anterior lens element 103 to assume more convex shape, thereby changing the diopter power of the lens.

The inner surface and thickness of anterior lens element 103 (relative to the optical axis of the lens) are selected so that the outer surface of anterior element 103 retains an optically corrective shape, e.g., spherical, throughout the entire range of motion of lens piston 104, e.g., for accommodations 0-10 diopters. It should of course be understood that the inner surface and thickness of anterior lens element 103 also may be selected to provide an aspherical outer surface, as required for a desired degree of optical correction.

Haptic portion 102 comprises bellows 107 and arms 108 and 109. Preferably, arms 108 and 109 are molded of a relatively rigid polymeric material, such as polyimide, that tends to return to its undeformed shape after a deformation occurs. Other embodiments of arms 108 and 109 may comprise shape memory material, such as nickel-titanium, or may be formed from other material having elastic properties. Bellows 107 preferably are an extension of substrate 105, and therefore preferably comprise light-transmissive materials, such as silicone or acrylic polymers or other biocompatible materials as are known in the art of intraocular lenses.

Each of arms 108 and 109 comprises a groove 110 that preferably extends from outer end 111 to bellows end 112. Arm mounting surface 113 of each of arms 108 and 109 is attached to bellows mounting surface 114 on bellows 107, preferably by bonding. Each of arms 108 and 109 further comprises notch 115 and cleat 116. Arms 108 and 109 extend outward some distance from optic portion 101, such that at least some portion of arms 108 and 109 contact the equator of capsule 15. Preferably, a portion of arms 108 and 109 near outer ends 111 remain in contact with inside wall of capsule 15 throughout the IOL's 100 full range of accommodation.

As discussed in detail below, as arms 108 and 109 move toward optic portion 101, bellows 107 contract and cause IOL 100 to accommodate. As arms 108 and 109 move away from optic portion 101, bellows 107 expand and cause IOL 100 to transition to an unaccommodated state. In the embodiment shown in FIG. 9, bellows 107 assume a contracted configuration when not acted upon by outside forces, such as forces on arms 108 and 109, thereby causing IOL 100 to be in a fully accommodated configuration. It should be recognized by one of skill in the art of intraocular lenses that IOL 100 could instead be configured to be in an unaccommodated configuration in its resting state by configuring bellows 107 to be fully expanded in the absence of outside forces.

Still referring to FIG. 9, bellows 107 are filled with a fluid such that movement of that fluid into and out of optic portion 101 changes the degree of accommodation of IOL 100 in a similar fashion as the above embodiments. In this regard, interiors of bellows 107 are in fluid communication with the interior of lens piston 104 via channels 117 that extend from bellows 107 to well 118 disposed beneath lens piston 104. Together, bellows 107, channel 117, well 118, lens piston 104, and fluid therein form a hydraulic system that deforms lens piston 104 responsive to movements of the ciliary muscle and the capsular bag that are transmitted through arms 108 and 109; these deformations of lens piston 104 in turn transition lens 100 between the accommodated and unaccommodated states.

In constructing the embodiment shown in FIG. 9 and described herein, intermediate layer 106 provides a barrier between channel 117 and anterior lens 103. Moreover, intermediate layer 106 has opening 119 in its center to permit fluid communication between lens piston 104 and well 118. Also, in addition to comprising bellows 107, substrate 105 further comprises posterior lens 120. It should be appreciated that the contacting surfaces between substrate 105 and intermediate layer 106 need not be planar, and may be curved convexly such that a failure of IOL 100 due to in a loss of fluid results in IOL 100 assuming a partially or fully accommodated state.

In accordance with one aspect of the present invention, IOL 100 is configured to be placed in the capsule of a patient's eye in the unaccommodated state, with the arms 108 and 109 in an undeflected position extending outward from optic portion 101. This configuration may be achieved by attaching suture 121 (one shown in dotted line attached to arm 109 in FIG. 9C) between notch 115 and cleat 116 along each of arms 108 and 109. When attached in this manner, suture 121 resides in grooves 110. Due to the tension placed on arms 108 and 109, relative movement of outer ends 111 is restricted, and arms 108 and 109 may be disposed against the interior wall of capsule 15. Optionally, barbs 122 may be disposed on arms 108 and 109 to facilitate affixation of arms 108 and 109 to the equator of capsule 15.

As shown in the enlarged portion of FIG. 9C, one embodiment of barbs in accordance with the present invention comprises two series of barbs 122 offset from each other. Barbs 122 are protrusions that are angled such that the outward edge protrudes at an acute angle, whereas the inward edge protrudes at a steeper angle, and may exceed ninety degrees. With this configuration, barbs 122 would not inhibit proper positioning into the capsular equator, but resists inward motion away from the interior surface of the capsular wall.

Sutures 121 preferably are attached prior to inserting IOL 100 into the patient's eye and may be released naturally due to a matching of resorbable characteristics to the healing period. Alternatively, the sutures could be released, for example, using a Nd:YAG laser or other known device.

When IOL 100 is implanted into the patient's native capsule 15 in the unaccommodated state, barbs 122 engage the capsular equator. Sutures 121 preferably remain in place during an initial period of several days to weeks, during which time fibrous tissue adheres to barbs 122, causing arms 108 and 109 to become affixed to the capsular equator. Preferably, sutures 121 comprise a resorbable material that disintegrates after several days to weeks, thereafter releasing arms 108 and 109 to allow IOL 100 to accommodate responsive to movements of the ciliary muscles and capsule.

More specifically, once sutures 122 are released or resorbed, arms 108 and 109 are free to flex and move inward under the bias of the predetermined shape of bellows 107 until constrained by the opposing forces applied by the ciliary muscles, capsule and zonules. For example, if the ciliary muscles contract, the capsular equator moves radially inward and arms 108 and 109 move inward, thereby permitting bellows 107 to collapse. This action displaces fluid from the interior of bellows 107 to lens piston 104, causing the anterior lens element to deflect to an accommodated state. When the ciliary muscles relax, the zonules pull the capsular equator to a larger diameter, thereby applying tensile forces to arms 108 and 109 that expand bellows 107. This action causes fluid in lens piston 104 to move to the interior of bellows 107, and reduces the degree of accommodation provided by IOL 100.

In a preferred embodiment, the resorbable characteristics of sutures 121 may be selected to correspond to the expected “heal-in” period, that is, the period during which fibrous growth cause barbs 122 to become embedded in the capsular equator. Thus, the sutures will naturally resorb and release arms 108 and 109, thereby allowing accommodating movement of lens piston 104 and anterior element 103.

As for the preceding embodiment, the volume of fluid in IOL 100 may be selected so that the forces required to provide a useable range of accommodation are satisfactory for a preselected population of patients. Alternatively, the volume of fluid used in IOL 100 may be specified during manufacture for a given patient, or may be adjusted prior to implantation of IOL 100 on a patient-by-patient basis. In this manner, the forces developed by lens piston 104 and the arms 108 and 109 may be tailored for a specific patient.

As will of course be understood, optic portion 101 could instead be arranged so that the lens piston is integrated with posterior lens element 120 instead of anterior lens element 103, and causes deflection the posterior lens element 120; the arrangement depicted in FIG. 9 is illustrative only.

While preferred illustrative embodiments of the invention are described above, it will be apparent to one skilled in the art that various changes and modifications may be made therein without departing from the invention. The appended claims are intended to cover all such changes and modifications that fall within the true spirit and scope of the invention.

What is claimed is:

1. An intraocular lens for implantation in a patient's eye having a ciliary muscle coupled to a capsule having an equator, the intraocular lens comprising:
a substrate defining a fluid channel;
a lens element operatively coupled to a lens piston, the lens piston coupled in fluid communication with the fluid channel;
a fluid disposed in the lens piston and fluid channel;
at least one haptic piston disposed in fluid communication with the fluid channel and configured to induce movement of the fluid to and from the lens piston responsive to movement of the ciliary muscle; and
means for retaining the haptic piston in a predetermined configuration for a period of time sufficient to permit a portion of the haptic piston to adhere to the equator of the capsule.

2. The intraocular lens of claim 1 further comprising means for affixing a portion of the haptic piston to the equator of the capsule.

3. The intraocular lens of claim 1 wherein the means for retaining comprises a suture.

4. The intraocular lens of claim 3 wherein the suture is bioabsorbable.

5. The intraocular lens of claim 3 wherein the intraocular lens further comprises a support ring carrying an arm.

6. The intraocular lens of claim 5 wherein the haptic piston further comprises a piston element disposed from the arm, a reservoir and a diaphragm separating the piston element from the reservoir.

7. The intraocular lens of claim 6 wherein the arm is biased radially inward in an unstressed state.

8. The intraocular lens of claim 5 wherein the arm further comprises an arched portion and the means for affixing comprises a plurality of barbs extending from the arched portion.

9. The intraocular lens of claim 1 wherein the fluid has a refractive index substantially the same as a refractive index of the lens element and the substrate.

10. The intraocular lens of claim 1 wherein the lens element is an anterior lens element.

11. The intraocular lens of claim 1 wherein the means for retaining comprises a retainer rod.

12. The intraocular lens of claim 11 wherein the retainer rod comprises a shape memory material.

13. The intraocular lens of claim 12 wherein the intraocular lens further comprises a peripheral portion carrying an arm and a retainer support.

14. The intraocular lens of claim 13 wherein the haptic piston further comprises a bellows coupled to a reservoir.

15. The intraocular lens of claim 14 wherein the arm is biased radially inward in an unstressed state.

16. The intraocular lens of claim 14 wherein the arm further comprises a distal portion and the means for affixing comprises a plurality ribs extending from the distal portion.

17. An intraocular lens for implantation in a patient's eye having a ciliary muscle coupled to a capsule having an equator, the intraocular lens comprising:
a substrate defining a fluid channel;
a lens element operatively coupled to a lens piston, the lens piston coupled in fluid communication with the fluid channel;
a fluid disposed in the lens piston and fluid channel;
at least one haptic piston disposed in fluid communication with the fluid channel and configured to induce movement of the fluid to and from the lens piston responsive to movement of the ciliary muscle; and
means for affixing a portion of the haptic piston to the equator of the capsule.

18. The intraocular lens of claim 17 further comprising means for retaining the haptic piston in a predetermined configuration for a period of time sufficient to permit a portion of the haptic piston to adhere to the equator of the capsule.

19. The intraocular lens of claim 18 wherein the means for retaining comprises a suture.

20. The intraocular lens of claim 19 wherein the suture is bioabsorbable.

21. The intraocular lens of claim 18 wherein the intraocular lens further comprises a support ring carrying an arm.

22. The intraocular lens of claim 21 wherein the haptic piston further comprises a piston element disposed from the arm, a reservoir and a diaphragm separating the piston element from the reservoir.

23. The intraocular lens of claim 22 wherein the arm is biased radially inward in an unstressed state.

24. The intraocular lens of claim 21 wherein the arm further comprises an arched portion and the means for affixing comprises a plurality barbs extending from the arched portion.

25. The intraocular lens of claim 17 wherein the fluid has a refractive index substantially the same as a refractive index of the lens element and the substrate.

26. The intraocular lens of claim 17 wherein the lens element is an anterior lens element.

27. The intraocular lens of claim 18 wherein the means for retaining comprises a retainer rod.

28. The intraocular lens of claim 27 wherein the retainer rod comprises a shape memory material.

29. The intraocular lens of claim 28 wherein the intraocular lens further comprises a peripheral portion carrying an arm and a retainer support.

30. The intraocular lens of claim 29 wherein the haptic piston further comprises a bellows coupled to a reservoir.

31. The intraocular lens of claim 31 wherein the arm is biased radially inward in an unstressed state.

32. The intraocular lens of claim 29 wherein the arm further comprises a distal portion and the means for affixing comprises a plurality ribs extending from the distal portion.

33. An intraocular lens for implantation in a patient's eye having a ciliary muscle coupled to a capsule having an equator, the intraocular lens comprising:
a substrate defining a fluid channel;
a lens element operatively coupled to a lens piston, the lens piston coupled in fluid communication with the fluid channel;
a fluid disposed in the lens piston and fluid channel;
at least one bellows disposed in fluid communication with the fluid channel;
a haptic arm operably coupled with the at least one bellows and configured to induce movement of the fluid to and from the lens piston responsive to movement of the ciliary muscle; and
means for retaining the haptic arm in a predetermined configuration for a period of time sufficient to permit a portion of the haptic arm to adhere to the equator of the capsule.

34. The intraocular lens of claim 33 further comprising means for affixing a portion of the haptic arm to the equator of the capsule.

35. The intraocular lens of claim 33 wherein the means for retaining comprises a suture.

36. The intraocular lens of claim 35 wherein the suture is bioabsorbable.

37. The intraocular lens of claim 36 wherein the haptic arm is biased radially inward in an unstressed state.

38. The intraocular lens of claim 36 wherein the haptic arm further comprises an arched portion and the means for affixing comprises a plurality of barbs extending from the arched portion.

39. The intraocular lens of claim 33 wherein the fluid has a refractive index substantially the same as a refractive index of the lens element and the substrate.

40. The intraocular lens of claim 33 wherein the lens element is an anterior lens element.

41. A method of implanting an intraocular lens comprising:
providing an intraocular lens having an optic portion and a haptic portion, the intraocular lens having accommodated state and an unaccommodated state, the intraocular lens biased to the accommodated state;
securing the intraocular lens in a fixed unaccommodated state;
implanting the intraocular lens into a capsule of a patient's eye;
waiting a predetermined period of time sufficient for the haptic portion to adhere to the capsule; and
releasing the intraocular lens from the fixed unaccommodated state so that the intraocular lens transitions between the accommodated and unaccommodated states responsive to movement of the capsule.

42. The method of claim 41 further comprising:
providing a suture for retaining the intraocular lens in the fixed unaccommodated state,
wherein securing the intraocular lens in the fixed unaccommodated state comprises applying the suture to the haptic portion.

43. The method of claim 41 further comprising:
providing a retaining rod for retaining the intraocular lens in the fixed unaccommodated state,
wherein securing the intraocular lens in the fixed unaccommodated state comprises inserting the retaining rod within the haptic portion.
